(19) United States
(12) Patent Application Publication
(10) Pub. No.: US 2026/0174724 A1
(43) Pub. Date: Jun. 25, 2026

(54) COMPOSITION FOR PREVENTING AND/OR IMPROVING BRAIN DYSFUNCTION

(71) Applicant: THE INSTITUTE OF BIOACTIVE PEPTIDES, Lexington, MA (US)

(72) Inventor: Tianyi MA, Lexington, MA (US)

(73) Assignee: THE INSTITUTE OF BIOACTIVE PEPTIDES, Lexington, MA (US)

(21) Appl. No.: 19/001,442

(22) Filed: Dec. 25, 2024

(51) Int. Cl. A61K 31/352; A61K 31/198; A61K 31/231; A61K 31/375; A61K 31/685; A61K 31/7012; A61K 31/7048; A61P 25/28

(52) U.S. Cl. CPC A61K 31/352; A61K 31/198; A61K 31/231; A61K 31/375; A61K 31/685; A61K 31/7012; A61K 31/7048; A61P 25/28

(57) ABSTRACT

A composition for preventing and/or improving brain dysfunction, especially Alzheimer's disease is provided. The composition includes dihydromyricetin or salts thereof, L-theanine, taurine, and cerebroside, which can enhance the intestinal stability and bioavailability of dihydromyricetin and prevent and improve brain dysfunction through multiple mechanisms such as anti-inflammation, inhibition of DNA methylation, elimination of hematoma, and removal of Aβ. In addition, the composition can be used in combination with monoclonal antibody drugs to reduce adverse reactions such as cerebral edema and cerebral hemorrhage that may occur during the treatment of Alzheimer's disease.

### TECHNICAL FIELD

The present invention relates to a composition for preventing and/or improving brain dysfunction, in particular to a composition for preventing and/or improving Alzheimer's disease.

### BACKGROUND

Alzheimer's disease (AD) is a progressive neurodegenerative disease; its main pathological features include neuronal loss in the cerebral cortex, neurofibrillary tangles, and the formation of senile plaques. The main mechanism of AD involves the deposition of β-amyloid protein (Aβ), abnormal phosphorylation of Tau protein, neuroinflammation, and oxidative stress. These factors interact with each other, leading to neuronal dysfunction and death, which in turn affects memory, cognitive, and behavioral functions.

At present, the strategies for treating AD mainly include drug therapy and non-drug therapy. In terms of drug therapy, monoclonal antibody drugs such as Aducanumab are used to target Aβ deposition, but these drugs may cause adverse reactions such as cerebral edema and cerebral hemorrhage, which limits their clinical application. Therefore, for the treatment of AD, in addition to traditional drug therapy, new auxiliary treatment methods need to be explored.

Looking into the future, the ideal therapeutic product should have the effect of improving the pathological process of AD, alleviating symptoms, or providing brain protection to avoid adverse reactions such as cerebral edema. As an auxiliary treatment product, it has the effect of slowing down or eliminating the adverse reactions caused by therapeutic drugs when used in combination with therapeutic drugs. In this field, natural products show great potential due to their safety and multi-target mechanism of action. However, many natural products have defects such as poor solubility, unstable physicochemical properties, and low bioavailability, which limit their therapeutic effects.

In view of the above-mentioned defects of existing AD treatment products and natural products that can improve the progression of AD, the present invention aims to enhance their potential in the treatment of Alzheimer's disease through a new natural product composition and provide a safe and effective product with both therapeutic and auxiliary therapeutic effects in order to play an important role in the treatment and management of AD.

### SUMMARY

In order to solve the problem that AD treatment products have obvious adverse reactions and natural product compositions generally have low bioavailability, the present invention provides a drug combination of natural products, which has preventive, therapeutic, and ameliorative effects, can improve the therapeutic effect, and increase safety and stability.

Specifically, the purpose of the present invention is to provide a composition for preventing and/or improving brain dysfunction, the composition including dihydromyricetin (DHM) or salts thereof, L-theanine, taurine, and cerebroside.

Preferably, the composition, in parts by weight, includes 10-40 parts of dihydromyricetin or salts thereof, 1-5 parts of L-theanine, 1-5 parts of taurine, and 1-10 parts of cerebroside.

Preferably, the composition, in proportion to mass, has a mass ratio of DHM or salts thereof to taurine of (2-40):1; and/or a mass ratio of DHM or salts thereof to L-theanine of (2-40):1; and/or a mass ratio of DHM or salts thereof to cerebroside of (1-40):1.

Preferably, the composition further includes one or more of ascorbic acid, spinosin, linoleic acid, or phosphatidylserine.

Preferably, the cerebroside is selected from one or more of sea cucumber cerebroside, Cordyceps sinensis cerebroside, Sterculia lychnophora cerebroside, or soybean cerebroside; preferably 1-O-β-D-glucosyl-(2S,3R,4E,8Z)-2-[(2-hydroxyoctadecanoyl)amino]-4,8-octadecadiene-1,3-diol and/or 1-O-β-D-glucosyl-(2S,3R,4E,8Z)-2-[(2-hydroxyhexadecanoyl)amino]-4,8-octadecadiene-1,3-diol; more preferably 1-O-β-D-glucosyl-(2S,3R,4E,8Z)-2-[(2-hydroxyoctadecanoyl)amino]-4,8-octadecadiene-1,3-diol.

Another object of the present invention is to provide a preparation for preventing and/or improving brain dysfunction, including dihydromyricetin or salts thereof, L-theanine, taurine, and cerebroside, and carriers and/or excipients.

In the case where the above-mentioned active ingredients are prepared into a single preparation, there is no special limitation, and 0.01-10 parts by weight of L-theanine, taurine, and cerebroside can be prepared for each 1 part by weight of dihydromyricetin or salts thereof, respectively. Based on the comprehensive consideration of effect and cost, L-theanine, taurine, and cerebroside are respectively preferred by 0.05-5 parts by weight and are more preferred by 0.05-0.3 parts by weight, relative to each 1 part by weight of dihydromyricetin or salts thereof.

Another object of the present invention is to provide an application of a composition in the preparation of a product for preventing and/or improving brain dysfunction, the composition including dihydromyricetin or salts thereof, L-theanine, taurine, and cerebroside.

The products of the present invention include food, beverages, medicine, health care products, or dietary supplements.

Another object of the present invention is to provide a method for preventing and/or improving brain dysfunction, including administering to a patient a composition including dihydromyricetin or a salt thereof, L-theanine, taurine, and cerebroside.

The brain dysfunction described in the present invention includes schizophrenia, bipolar disorder, attention deficit disorder, attention deficit hyperactivity disorder, Alzheimer's disease, and Parkinson's disease, and related clinical symptoms include learning difficulties, memory loss, inability to concentrate, emotional apathy, emotional despair, irritability, loss of interest, lack of pleasure, sleep disorders, fatigue, decreased or increased appetite, weight loss or weight gain, personality changes, and lack of motivation.

In the present invention, “improvement” refers to the improvement of the symptoms and conditions of the disease, the prevention or delay of the symptoms and the deterioration of conditions of the disease, the reversal, prevention, or delay of the development of the symptoms of the disease, or the treatment of the disease. “Prevention” refers to the prevention of the onset of the disease in the application object, or the delay of the onset, or the reduction of the risk of the onset of the disease in the application object.

In the present invention, “treatment” refers to any treatment of the disease (e.g., improvement, alleviation, inhibition of progression, etc.). In addition, it also includes preventing the onset and/or progression of the disease.

In the present invention, “patient” refers to humans and other animals, such as dogs, cats, horses, etc. The patient is preferably a mammal, more preferably a human.

The purpose of the present invention is also to provide a use of a composition including dihydromyricetin or salts thereof, L-theanine, taurine, and cerebroside in the preparation of a product for eliminating cerebral hematoma. In particular, the use in the preparation of a product for eliminating cerebral hematoma caused by the administration of Alzheimer's disease monoclonal antibody drugs.

The core components of the present invention can be extracted, separated, and purified from animals and plants or directly adopted from dried powders or extract mixtures of animals and plants. For example, dihydromyricetin is mainly derived from plants with a high dihydromyricetin content, such as Ampelopsis grossedentata or Hovenia dulcis; L-theanine is mainly derived from tea leaves with a high theanine content, such as albino tea and yellow tea; taurine can be derived from marine plants and animals, animal viscera; cerebrosides are mainly derived from brain tissue, Cordyceps sinensis, soybeans, sea cucumbers, starfish, Linum usitatissimum, Sterculia lychnophora, and other animals and plants. For example, one or more of sea cucumber cerebroside, Cordyceps sinensis cerebroside, Sterculia lychnophora cerebroside, or soybean cerebroside; preferably 1-O-β-D-glucosyl-(2S,3R,4E,8Z)-2-[(2-hydroxyoctadecanoyl)amino]-4,8-octadecadiene-1,3-diol and/or 1-O-β-D-glucosyl-(2S,3R,4E,8Z)-2-[(2-hydroxyhexadecanoyl)amino]-4,8-octadecadiene-1,3-diol.

In addition, the core components of the present invention can also be derived from total synthesis or semi-synthesis by biological or chemical methods.

As long as it does not hinder the effect of preventing and/or improving brain dysfunction, the composition of the present invention can also be appropriately combined with other active ingredients (such as substances with auxiliary effects) and/or additives by methods known per se.

Other active ingredients that can be incorporated into the composition of the present invention include, for example, A vitamins; carotenoids (excluding lutein); B vitamins; C vitamins; D vitamins; E vitamins; tocotrienols; glutathione and its derivatives or salts thereof, polyphenols such as spinosin, catechins, anthocyanins, tannins, rutin, isoflavones, chlorogenic acid, ellagic acid, curcumin, and coumarin; linoleic acid, α- or γ-linolenic acid, arachidonic acid, eicosapentaenoic acid, docosahexaenoic acid, and derivatives or salts thereof; proteins selected from collagen, elastin, fibronectin, and keratin, and derivatives and hydrolyzates thereof; α-hydroxy acids such as glycolic acid, lactic acid, malic acid, citric acid, and salicylic acid, and corresponding derivatives or salts thereof, yeast, lactic acid bacteria, bifidobacteria, Ziziphus jujuba seeds, Ganoderma lucidum, ginseng, rosemary, and Phellodendron chinense, garlic, hinokitiol, cepharanthine, aloe, sage, Arnica montana, chamomile, white birch, Hypericum perforatum, eucalyptus, Millettia reticulata, Chamaecrista mimosoides, white Mulberry Root-bark, Chinese Angelica, Lightyellow sophora root, hawthorn fruit, coix seed, Houttuynia cordata, seaweed, natto, lemongrass, ginkgo leaf extract, phosphatidylserine and other natural substances or their extracts; adenosine triphosphate, adenosine diphosphate, adenosine monophosphate and other adenylic acid derivatives; iron, vanadium, molybdenum, manganese, copper, potassium, magnesium, calcium, zinc, selenium, iodine and other minerals; mannitol, xylitol, glucosamine and other monosaccharides; hyaluronic acid, chondroitin sulfate, dermatan sulfate, heparan sulfate, heparin, keratan sulfate, glycogen, chitin, chitosan and other polysaccharides; deoxyribonucleic acid, ribonucleic acid and other nucleic acids; glycyrrhizic acid, guanine, mucin, ubiquinone, α-lipoic acid, octacosanol, allysine, alliin, and other substances and mixtures thereof.

Relative to the total weight of the composition, other active ingredients can be formulated in an amount of, for example, 0.1-99% by weight, preferably 1-90% by weight, more preferably 5-80% by weight, further preferably 10-70% by weight, and particularly preferably 20-60% by weight. Two or more other active ingredients can also be used in combination.

The composition of the present invention is formulated and administered by a route selected from the following: oral, injection, rectal, nasal, pulmonary, topical, oral and sublingual, vaginal, parenteral, subcutaneous, intramuscular, intravenous, intradermal, intrathecal, and epidural.

The composition is preferably taken orally. The oral dosage form is not particularly limited, and any oral dosage form known in the art can be used, preferably including tablets, capsules, suspensions or oral solutions, and other oral dosage forms known in the art. When used as an oral dosage form, the dosage standard used is, for example, 500-1500 mg/day, preferably 700-1200 mg/day, preferably 800-1000 mg/day, and most preferably 1000 mg/day.

The duration of administration of the composition of the present invention may depend on the severity of the disease, preferably at least 1 month, for example, 1, 2, 3, 4, 5, or 6 months, and may be taken for life as required by the disease.

According to embodiments of the present invention, the composition may also contain pharmaceutically acceptable excipients; preferably, the excipients are selected from at least one of the following excipients, including but not limited to: fillers, disintegrants, binders, lubricants, surfactants, flavoring agents, wetting agents, pH regulators, solubilizers or cosolvents, and osmotic pressure regulators. Those skilled in the art can easily determine how to select the corresponding excipients and their corresponding dosages according to the needs of the specific dosage form.

### Beneficial Effects

The composition of the present invention can enhance the stability of dihydromyricetin in the intestinal environment and improve the bioavailability of dihydromyricetin.

The composition of the present invention can prevent and improve brain dysfunction through multiple mechanisms such as anti-inflammation, inhibition of DNA methylation, elimination of hematoma, and gentle removal of Aβ.

The composition of the present invention is expected to be used in combination with monoclonal antibody drugs for the treatment of AD while assisting in the removal of Aβ, reducing the adverse reactions caused by antibody drugs, such as cerebral edema (amyloid-related imaging abnormalities-edema, ARIA-E), cerebral hemorrhage (amyloid-related imaging abnormalities-hemorrhage, ARIA-H), and inflammation.

### BRIEF DESCRIPTION OF THE DRAWINGS

FIG. 1: Stability experiment of DHM and its combination in SIF.

FIG. 2: Stability experiment of DHM and its combination in SIF+Fe³⁺.

FIG. 3: Hematoma volume on the first day of modeling.

FIG. 4: Hematoma volume on the third day of modeling.

FIG. 5: AGEs content detection.

FIG. 6: Aβ1-42 content detection.

FIG. 7: IL-1ß content detection.

FIG. 8: TNF-α content detection.

FIG. 9: IL-6 content detection.

FIG. 10: DNMT1 activity determination.

### TERM DEFINITIONS AND EXPLANATIONS

Unless otherwise specified, the definitions of groups and terms recorded in the specification and claims of this application, including their definitions as examples, exemplary definitions, preferred definitions, definitions recorded in tables, definitions of specific compounds in the embodiments, etc., can be arbitrarily combined and bonded with each other. The definitions of groups and compound structures after such combination and bonding should be understood to be within the scope of the specification and/or claims of this application.

In this application, the term “salt” or “pharmaceutically acceptable salt” includes pharmaceutically acceptable acid addition salts and pharmaceutically acceptable base addition salts. The term “pharmaceutically acceptable” refers to those compounds, materials, compositions, and/or dosage forms that are suitable for use in contact with human and animal tissues within the scope of reliable medical judgment without excessive toxicity, irritation, allergic reactions, or other problems or complications and are commensurate with a reasonable benefit/risk ratio.

References to “one embodiment,” “embodiment,” etc. in the specification indicate that the described embodiment may include specific aspects, features, structures, parts, or characteristics, but not every embodiment must include such aspects, features, structures, parts, or characteristics. In addition, such phrases may but do not necessarily refer to the same embodiment mentioned in other parts of the specification. In addition, when a specific aspect, feature, structure, part, or characteristic is described in conjunction with an embodiment, whether or not explicitly described, it is within the knowledge of those skilled in the art to influence or associate that aspect, feature, structure, part, or characteristic with other embodiments.

As understood by those skilled in the art, all numerical values, including those expressing the amounts of ingredients, properties such as molecular weight, reaction conditions, etc., are approximate and are understood to be optionally modified in all cases by the term “about.” These values may vary depending on the desired properties sought to be obtained by those skilled in the art utilizing the teachings described herein. It is also understood that these values inherently contain variability that necessarily results from the standard deviation found in their respective test measurements.

Whenever the term “including” is used herein, the option of using the term “consisting of” or “including or consisting essentially of” is contemplated. As used herein, “comprising” is synonymous with “including” or “characterized by” and is inclusive or open-ended and does not exclude additional, unlisted elements or method steps. As used herein, “consisting of” excludes any element, step, or ingredient not specified in the aspect element. As used herein, “consisting essentially of” does not exclude materials or steps that have no substantial effect on the basic and novel characteristics of the aspect. In each embodiment herein, any of the terms “including,” “consisting essentially of,” and “comprising” may be replaced with either of the other two terms. The disclosure illustratively described herein may suitably be practiced without limitation or limitation by any one or more elements not specifically disclosed herein.

### DETAILED DESCRIPTION OF THE EMBODIMENTS

The following embodiments are intended to illustrate the above invention and should not be construed as narrowing its scope. Those skilled in the art will readily appreciate that the embodiments propose many other ways that can implement the present invention. It should be understood that while remaining within the scope of the present invention, many changes and modifications can be made.

### Embodiment 1 Stability Experiment

Dihydromyricetin (DHM) is relatively stable in an acidic environment with a pH value of 3-6 but is easily decomposed in an alkaline environment. In simulated intestinal fluid (SIF), DHM undergoes a degradation process similar to first-order kinetics. The presence of metal ions such as Fe³⁺ can accelerate the oxidation process of DHM, thereby reducing its stability in intestinal fluid. The purpose of this embodiment is to study the effect of the interaction of the core components of the composition of the present invention on the stability of DHM.

### Experimental Plan:

Solutions A-D and A1-D1 were prepared according to the following components and concentrations, with a volume of 10 mL each. The mixed solution was placed in a water bath at 37° C., and the content of the remaining DHM was determined by HPLC at different time intervals.
Solution A: SIF+DHM (1 mg/mL);
Solution B: SIF+DHM (1 mg/mL)+taurine (0.3 mg/mL)
Solution C: SIF+DHM (1 mg/mL)+L-theanine (0.4 mg/mL);
Solution D: SIF+DHM (1 mg/mL)+taurine (0.1 mg/mL)+L-theanine (0.1 mg/mL)
Solution A1: SIF+DHM (1 mg/mL)+Fe³⁺ (50 μmol/L);
Solution B1: SIF+DHM (1 mg/mL)+taurine (0.3 mg/mL)+Fe³⁺ (50 μmol/L);
Solution C1: SIF+DHM (1 mg/mL)+L-theanine (0.4 mg/mL)+Fe³⁺ (50 μmol/L);
Solution D1: SIF+DHM (1 mg/mL)+taurine (0.1 mg/mL)+L-theanine (0.1 mg/mL)+Fe³⁺ (50 μmol/L).

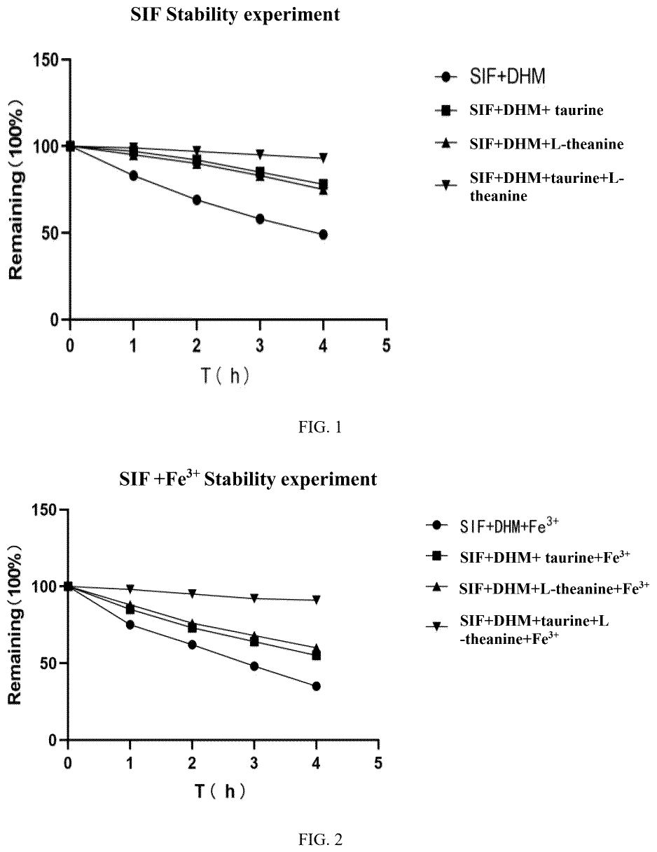

According to the data in FIG. 1, dihydromyricetin (DHM) is easily degraded in simulated intestinal fluid (SIF). After 4 h of incubation, the remaining amount of DHM dropped below 50%. The experiment found that when only taurine or L-theanine was added to the formula, the degradation process of DHM could be delayed, and when taurine and L-theanine were included in the formula at the same time, the degradation of DHM could be greatly delayed.

FIG. 2 further shows that the degradation rate of DHM is accelerated when Fe³⁺ is present in the SIF solution. In this case, the delay effect of adding taurine or L-theanine alone is not obvious, but if taurine and L-theanine are added at the same time, the degradation of DHM can still be significantly delayed, so that the retention of DHM after 4 h of incubation exceeds 90%.

In addition, the experiment also studied the effect of different amounts of taurine and L-theanine on delaying the degradation of DHM in the presence of Fe³⁺. The results showed that when the mass ratio of DHM, taurine, and L-theanine was controlled in the range of 10-20:1-5:1-5, the degradation of DHM in SIF could be significantly delayed, ensuring that the retention of DHM after 4 h of incubation was maintained at more than 90%.

### Embodiment 2 Bioavailability Experiment

Dihydromyricetin (DHM) is not highly soluble in water and is unstable in an alkaline environment, which limits its oral bioavailability. This experiment aims to evaluate the potential effect of the composition of the present invention on improving the bioavailability of DHM.

### Experimental Design:

Experimental subject grouping: 24 male SD rats, weighing between 200-220 g, were randomly divided into four groups.

### Dosage Regimen:

Group A (control group): DHM was administered alone at a dose of 100 mg/kg.
Group B: DHM (100 mg/kg) was co-administered with L-theanine (10 mg/kg).
Group C: DHM (100 mg/kg) was co-administered with taurine (10 mg/kg).
Group D: DHM (100 mg/kg), L-theanine (10 mg/kg), and taurine (10 mg/kg) were co-administered.

### Administration Method:

The drug was mixed with 3 mL of 0.5% CMC-Na solution and administered by gavage.

### Sample Collection and Processing:

At 0.1, 0.5, 1, 1.5, 2, 2.5, 3, 4, 6, 8, and 12 h after administration, 200 μL of blood samples were collected through the tail vein and placed in heparinized EP tubes.

The blood samples were centrifuged at 5000 rpm for 15 min at 4° C., and plasma was collected and stored at −20° C.

Before analysis, 30 μL of plasma sample was taken, 30 μL of internal standard (rutin) solution and 140 μL of 1% formic acid-acetonitrile solution were added, vortex oscillated for 1 min, centrifuged at 12000 rpm for 5 min, and 150 μL of supernatant was taken for LC-MS/MS analysis. Analysis purpose:

The concentration of DHM in plasma was determined at different time points to evaluate the effect of the composition on the bioavailability of DHM.

TABLE 1
Bioavailability experiment

| Parameter | Group A | Group B | Group C | Group D |
|---|---|---|---|---|
| Cₘₐₓ (ng/mL) | 118.35 ± 13.67 | 234.64 ± 22.47 | 217.82 ± 34.12 | 364.53 ± 101.76 |
| tₘₐₓ (h) | 0.83 ± 1.12 | 0.89 ± 0.29 | 0.86 ± 0.75 | 0.94 ± 0.63 |
| t_{1/2} (h) | 1.91 ± 0.67 | 3.59 ± 0.72 | 3.48 ± 0.58 | 4.82 ± 0.63 |
| AUC_{0→t} | 10.35 ± 0.29 | 20.43 ± 0.46 | 19.02 ± 0.27 | 31.74 ± 1.06 |
| (mg/L · min) | | | | |
| AUC_{0→∞} | 11.63 ± 0.61 | 21.24 ± 0.67 | 19.87 ± 0.95 | 32.35 ± 0.75 |
| (mg/L · min) | | | | |
| CL (L/min/kg) | 8.62 ± 1.04 | 4.74 ± 0.46 | 5.06 ± 0.71 | 3.12 ± 0.62 |

According to the data in Table 1:

Improved bioavailability: Compared with group A (administration of DHM alone), key pharmacokinetic parameters such as the Cₘₐₓ (maximum plasma concentration), t_{1/2} (half-life), AUC_{0→t} (area under the curve of administration to a specific time), AUC_{0→∞} (area under the curve of administration to infinite time), and CL (clearance rate) of groups B, C, and D were significantly improved. This indicates that adding L-theanine and/or taurine can significantly improve the bioavailability of DHM.

Combined effect: Group D (DHM combined with L-theanine and taurine) showed the best effect on all pharmacokinetic parameters. The Cₘₐₓ of group D was more than 3 times that of group A, t_{1/2} was more than 2.5 times that of group A, AUC_{0→∞} was also significantly higher than that of group A, while CL was significantly lower than that of group A. These results show that DHM in group D is better than other groups in terms of absorption, distribution, metabolism, and excretion in the body.

Statistical significance: Compared with groups B and C, group D also showed statistically significant differences in parameters such as Cₘₐₓ, t_{1/2}, AUC_{0→∞}, and CL (P<0.01). This further confirms the superiority of the combined dosing regimen of DHM with L-theanine and taurine in improving bioavailability.

### Embodiment 3 Cerebral Hematoma Experiment

In the treatment of Alzheimer's disease (AD), the use of monoclonal antibody drugs may cause amyloid-related imaging abnormalities (ARIA), which is a problem that needs to be strictly monitored during the treatment process. Studies have shown that these drugs can quickly clear β-amyloid protein (Aβ) in a short period of time, which may cause Aβ deposition on the blood vessel wall, thereby affecting the integrity of the blood vessels and ultimately leading to the occurrence of ARIA. The main symptoms of ARIA include cerebral edema and cerebral hemorrhage, and the occurrence of these symptoms is directly related to the dosage and frequency of drug administration. The purpose of this experiment is to simulate and evaluate the effect of the composition of the present invention on cerebral hematoma, which is of great significance for improving AD treatment regimens and reducing potential risks during treatment.

This experiment aims to evaluate the effect of different compositions on the hematoma volume in the intracerebral hemorrhage (ICH) model of C57BL/6N mice. A total of 72 male C57BL/6N mice, aged 8-10 weeks, were randomly divided into 6 groups and treated with gavage for 7 days. Subsequently, except for the sham operation group (CON), the remaining groups were induced the ICH model by collagenase method. The experimental groups and treatments are as follows:
CON group: gavage with CMC-Na solution for 7 days, followed by injection of PBS in the basal ganglia region.
MOD group: gavage with CMC-Na solution for 7 days, followed by injection of collagenase in the basal ganglia region, as the model control group.
G1 group: gavage with DHM (100 mg/kg·d) for 7 days, followed by injection of collagenase in the basal ganglia region.
G2 group: gavage with DHM (50 mg/kg·d), L-theanine (5 mg/kg·d) and taurine (5 mg/kg·d) for 7 days, followed by injection of collagenase in the basal ganglia region.
G3 group: gavage with DHM (30 mg/kg·d), L-theanine (3 mg/kg·d), taurine (3 mg/kg·d), and cerebroside (3 mg/kg·d) for 7 days, followed by injection of collagenase in the basal ganglia region.

G4 group: gavage with L-theanine (5 mg/kg·d), taurine (5 mg/kg·d), and cerebroside (10 mg/kg·d) for 7 days, followed by injection of collagenase in the basal ganglia region.

On the 1st and 3rd day after modeling, 6 mice were randomly selected from each group for decapitation and brain sampling. The brain tissue was cut into slices on ice, and the hematoma volume was calculated using ImageJ software.

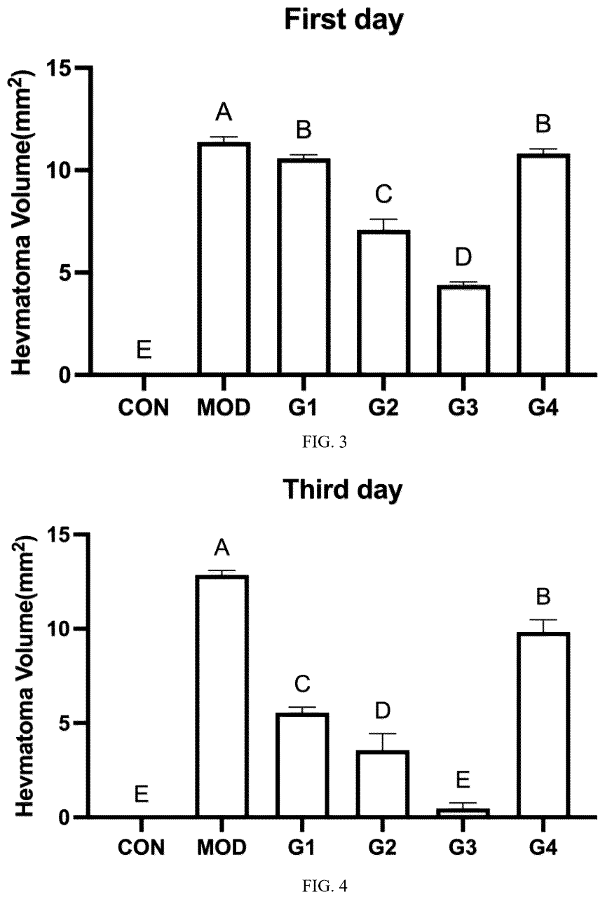

Based on the hematoma volume measurement results on the 1st and 3rd days, the following conclusions can be drawn (as shown in FIGS. 3-4):

Day 1: Compared with the MOD group, the hematoma volume of the G1 and G4 groups decreased, while the hematoma volume of the G2 and G3 groups decreased more significantly, indicating that these compositions can reduce the hematoma caused by ICH to a certain extent.

Day 3: The hematoma volume of all treatment groups decreased significantly compared with the MOD group, among which the effect of the G3 group was the most significant, which may mean that the composition of the G3 group has potential therapeutic effects in reducing the hematoma caused by ICH.

### Embodiment 4 Brain Function Improvement Experiment

Alzheimer's disease (AD) is a complex neurodegenerative disease, and its pathogenesis involves multiple factors that jointly promote the pathological process of AD. This embodiment will evaluate the effects of different compositions on the pathological process of AD and their potential effects on improving brain function. By comparing the biomarkers in the brain tissue of each group of rats, such as AGEs, Aβ1-42, inflammatory factors (IL-1B, TNF-α, IL-6), and DNA methyltransferase (DNMT1) activity, the improvement effect of the composition on the pathological characteristics of AD can be evaluated. In addition, the experiment will also explore the effects of different compositions on the cognitive function of rats to provide a scientific basis for the development of new AD treatment strategies.

### Experimental Plan

A total of 36 healthy male SD rats aged 6-8 weeks, weighing between 200-220 g, were selected and randomly divided into 6 groups for an 8-week experiment.
CON group (normal control group): Rats were fed with normal saline according to body weight and injected with normal saline subcutaneously in the back of the neck as normal controls.
MOD group (model group): rats were gavaged with normal saline according to body weight, and 200 mg/kg of D-galactose saline solution was subcutaneously injected at the back of the neck to induce the AD model.
G1 group (treatment group 1): rats were gavaged with normal saline suspension containing DHM (50 mg/kg·d), L-theanine (5 mg/kg·d), and taurine (5 mg/kg·d) according to body weight, and 200 mg/kg of D-galactose saline solution was subcutaneously injected at the back of the neck.
G2 group (treatment group 2): rats were gavaged with normal saline suspension containing DHM (20 mg/kg·d), L-theanine (3 mg/kg·d), taurine (3 mg/kg·d) and cerebroside (5 mg/kg·d) according to body weight, and 200 mg/kg of D-galactose saline solution was subcutaneously injected at the back of the neck.
Group G3 (treatment group 3): The rats were fed with Hovenia dulcis powder (containing 50 mg DHM), L-theanine (4 mg), taurine (4 mg), and Sterculia lychnophora powder (containing 1 mg cerebroside) and gavaged with normal saline according to body weight. A 200 mg/kg D-galactose saline solution was subcutaneously injected at the back of the neck.
Group G4 (treatment group 4): The rats were gavaged with a normal saline suspension containing L-theanine (10 mg/kg·d), taurine (10 mg/kg·d), and cerebroside (10 mg/kg·d) according to body weight. A 200 mg/kg D-galactose saline solution was subcutaneously injected at the back of the neck.

After the experiment, all rats were fasted for 24 h except for water. They were then anesthetized, weighed, and perfused with normal saline to fix the brain tissue. The separated brain tissue was placed in a container and frozen in an ultra-low temperature freezer at −80° C. for subsequent analysis.

### Aβ1-42 and AGEs

Aβ1-42 is one of the key pathological features of Alzheimer's disease (AD). It originates from the β- and γ-secretase cleavage of amyloid precursor protein (APP). This protein fragment accumulates in the brain to form amyloid plaques, which is one of the most typical pathological changes of AD. The deposition of amyloid plaques is closely related to neuronal dysfunction and death, which in turn affects memory, cognitive, and behavioral functions.

On the other hand, abnormal advanced glycation end products (AGEs) accumulate in the brains of AD patients, which may be involved in the pathogenesis of AD by impairing neuronal function and promoting oxidative stress. The accumulation of AGEs is associated with neurodegenerative processes, and they may affect the health of neurons through multiple pathways, including altering cell signaling, promoting inflammatory responses, and interfering with normal cellular metabolic processes.

In summary, the deposition of Aβ1-42 and the accumulation of AGEs play an important role in the pathological process of AD. They not only reflect the core pathological features of the disease but may also be important factors driving the progression of the disease. Therefore, research on these pathological features and the development of therapeutic strategies are crucial for the management and treatment of AD.

About 0.02 g of tissue samples were taken from the same part of the brain tissue of each rat, and the content of AGEs and Aβ1-42 in the brain tissue was detected according to the instructions of the commercial kit (Abcam AGE (Advanced Glycation End) Assay Kit; Thermo Fisher Scientific Human Amyloid beta 42 ELISA Kit).

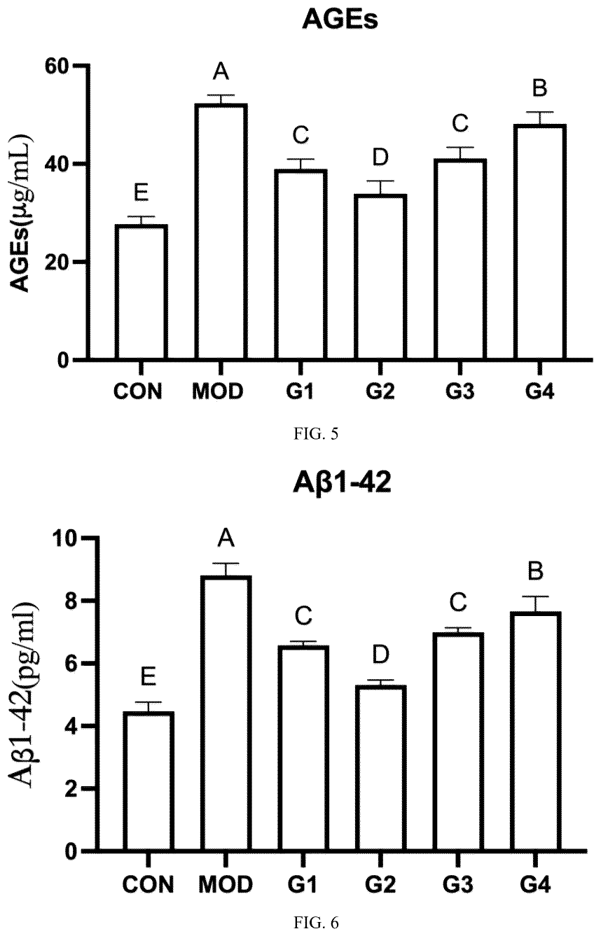

According to the data in FIGS. 5-6, the levels of AGEs and Aβ1-42 in the brain tissue of rats in the AD model group (MOD) were significantly increased compared with the normal control group (CON), which is consistent with the pathological characteristics of AD. Compared with the model group, the levels of AGEs and Aβ1-42 in all treatment groups decreased significantly, showing the positive effect of treatment.

In particular, the reduction effect of AGEs and Aβ1-42 in treatment group 2 (G2) after the addition of cerebrosides was the most significant, indicating that the composition has a potential therapeutic effect in reducing AD-related biomarkers. This may be related to the neuroprotective effect of cerebrosides, which helps to improve the pathological process of AD.

### IL-1β, TNF-α, IL-6

IL-1β, TNF-α, and IL-6 play an important role in Alzheimer's disease (AD). They are cytokines closely related to the inflammatory response in the pathological process of AD.

IL-1β is a multifunctional immunomodulatory cytokine. Its level in the cerebrospinal fluid and serum of AD patients is significantly increased, and it is overexpressed in the early stage of plaque formation. IL-1ß can promote the transformation of diffuse amyloid plaques into neuritic plaques and assist these neuritic plaques to spread in the cortex. It can also produce more Aβ by promoting APP degradation and activating microglia, leading to neuronal damage.

TNF-α is produced by microglia after stimulation. It can not only significantly activate cultured astrocytes but also promote the release of TNF-α, nitric oxide, glutamate, and other substances, which have toxic effects on neurons. TNF-α can stimulate monocytes to produce IL-6 and IL-1β and can also induce IL-8 and induce microglia to synthesize colony-stimulating factors, further amplifying inflammation.

IL-6 is mainly produced by mononuclear macrophages, Th2 cells, vascular endothelial cells, fibroblasts, and glial cells after inflammation or other stimulation. IL-6 is related to the synthesis of APP, and there is a large amount of IL-6 in the senile plaques of AD patients, and its expression precedes the formation of senile plaques. Overexpression of IL-6 can lead to neuronal degeneration and accelerate the formation of neurofibrillary tangles.

These inflammatory factors not only participate in the inflammatory response of AD but also promote the progression of AD by affecting the production and deposition of Aβ, the phosphorylation of Tau protein, and the damage and death of neurons. Therefore, they play a key role in the pathogenesis of AD and may become potential targets for the treatment of AD.

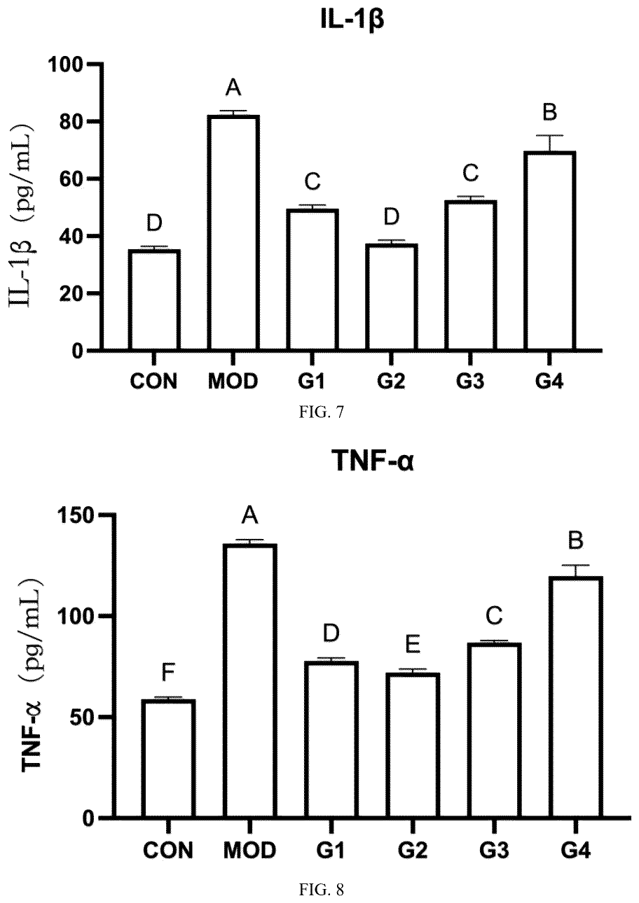

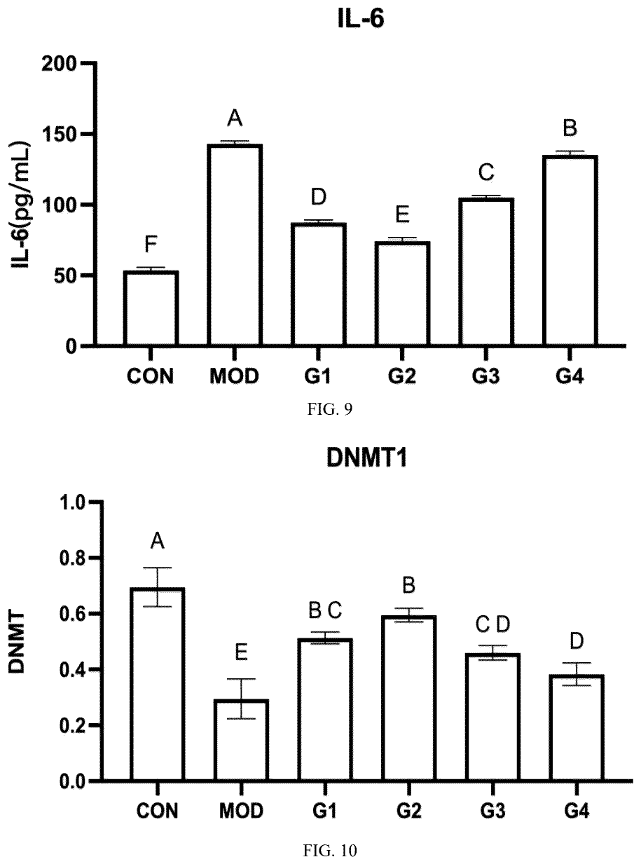

As shown in FIGS. 7-9, in this experiment, we used enzyme-linked immunosorbent assay (ELISA) to quantitatively analyze the inflammatory-related factors IL-1β, TNF-α, and IL-6 in the hippocampus region. The experimental results revealed that the levels of these inflammatory factors in D-galactose-induced AD model rats (MOD group) were significantly increased compared with the normal control group (CON group), which is consistent with the characteristics of inflammatory response in AD pathology.

Specifically, the levels of IL-1β, TNF-α, and IL-6 in the MOD group were significantly increased compared with those in the CON group, indicating that D-galactose successfully induced an inflammatory response. However, compared with the MOD group, the levels of these inflammatory factors in all treatment groups (G1-G4) were significantly reduced, showing a positive effect of the treatment.

It is particularly noteworthy that treatment group 2 (G2) had the most significant effect in reducing the levels of IL-1β, TNF-α, and IL-6, and its levels were close to or even lower than those of the normal control group (CON group). This suggests that the treatment regimen of group G2 has a potential therapeutic effect in inhibiting AD-related inflammatory responses.

### DNMT1 Activity Assay

DNMT1 is a key DNA methyltransferase responsible for maintaining DNA methylation patterns during the cell cycle. In the pathogenesis of AD, changes in the expression of DNMT1 may play an important role. In particular, the accumulation of Aβ1-42 has been shown to reduce the expression of DNMT1 protein in brain cells, which may interfere with the normal DNA methylation process, thereby affecting gene expression and nerve cell function.

In order to evaluate the expression level of DNMT1, this experiment used the EpiQuik extraction kit to extract proteins from brain tissue and the EpiQuik DNMT1 Assay Kit to measure the activity of DNMT1. The percentage inhibition of DNMT1 activity was calculated by comparing the experimental results with the control DNMT1 activity provided in the kit.

The experimental results (shown in FIG. 10) showed that compared with the normal control group (CON group), the expression of DNMT1 protein in the brain tissue of the AD model group (MOD group) was significantly reduced. This is consistent with the expectation of Aβ1-42-induced decrease in DNMT1 expression. However, compared with the model group, the DNMT1 protein expression levels in all treatment groups (G1-G4 groups) were significantly increased, indicating that treatment can promote the recovery of DNMT1. In particular, treatment group 2 (G2 group) showed the most significant increase in DNMT1 expression, which may be related to its significant effect in reducing inflammatory factors.

While specific embodiments have been described above with reference to disclosed embodiments and examples, such embodiments are illustrative only and do not limit the scope of the invention. Changes and modifications may be made by one of ordinary skills in the art without departing from the broader aspects of the invention as defined by the appended claims. All publications, patents, and patent documents are hereby incorporated by reference to the same extent as if individually incorporated by reference. Limitations inconsistent with this disclosure should not be construed thereby. The invention has been described with reference to various specific and preferred embodiments and techniques. However, it will be understood that many changes and modifications can be made while remaining within the spirit and scope of the invention.

What is claimed is:

1. A composition for preventing and/or improving a brain dysfunction, comprising dihydromyricetin (DHM) or a salt of the DHM, L-theanine, taurine, and cerebroside.

2. The composition for preventing and/or improving the brain dysfunction according to claim 1, wherein, in parts by weight, the composition comprises: 10-40 parts of the DHM or the salt of the DHM, 1-5 parts of the L-theanine, 1-5 parts of the taurine, and 1-10 parts of the cerebroside.

3. The composition for preventing and/or improving the brain dysfunction according to claim 1, wherein a mass ratio of the DHM or the salt of the DHM to the taurine is (2-40):1; and/or a mass ratio of the DHM or the salt of the DHM to the L-theanine is (2-40):1; and/or a mass ratio of the DHM or the salt of the DHM to the cerebroside is (1-40):1.

4. The composition for preventing and/or improving the brain dysfunction according to claim 1, further comprising one or more of ascorbic acid, spinosin, linoleic acid, or phosphatidylserine.

5. The composition for preventing and/or improving the brain dysfunction according to claim 1, wherein the cerebroside is selected from one or more of sea cucumber cerebroside, Cordyceps sinensis cerebroside, Sterculia lychnophora cerebroside, or soybean cerebroside.

6. A preparation for preventing and/or improving a brain dysfunction, comprising the composition according to claim 1.

7. The preparation for preventing and/or improving the brain dysfunction according to claim 6, further comprising a carrier and/or an excipient.

8. A method for preventing and/or improving a brain dysfunction, comprising administrating to a subject an effective amount of the composition according to claim 1.

9. The method according to claim 8, wherein the brain dysfunction comprises: schizophrenia, bipolar disorder, attention deficit disorder, attention deficit hyperactivity disorder, cerebral hematoma, Alzheimer's disease, and Parkinson's disease.

10. A method for preventing and/or improving a brain dysfunction, comprising administering to a patient the composition according to claim 1 or a preparation, wherein the preparation comprises the composition.

11. The composition for preventing and/or improving the brain dysfunction according to claim 2, further comprising one or more of ascorbic acid, spinosin, linoleic acid, or phosphatidylserine.

12. The composition for preventing and/or improving the brain dysfunction according to claim 3, further comprising one or more of ascorbic acid, spinosin, linoleic acid, or phosphatidylserine.

13. The composition for preventing and/or improving the brain dysfunction according to claim 2, wherein the cerebroside is selected from one or more of sea cucumber cerebroside, Cordyceps sinensis cerebroside, Sterculia lychnophora cerebroside, or soybean cerebroside.

14. The composition for preventing and/or improving the brain dysfunction according to claim 3, wherein the cerebroside is selected from one or more of sea cucumber cerebroside, Cordyceps sinensis cerebroside, Sterculia lychnophora cerebroside, or soybean cerebroside.

15. The method according to claim 8, wherein in parts by weight, the composition comprises: 10-40 parts of the DHM or the salt of the DHM, 1-5 parts of the L-theanine, 1-5 parts of the taurine, and 1-10 parts of the cerebroside.

16. The method according to claim 8, wherein in the composition, a mass ratio of the DHM or the salt of the DHM to the taurine is (2-40):1; and/or a mass ratio of the DHM or the salt of the DHM to the L-theanine is (2-40):1; and/or a mass ratio of the DHM or the salt of the DHM to the cerebroside is (1-40):1.

17. The method according to claim 10, wherein in parts by weight, the composition comprises: 10-40 parts of the DHM or the salt of the DHM, 1-5 parts of the L-theanine, 1-5 parts of the taurine, and 1-10 parts of the cerebroside.

18. The method according to claim 10, in the composition, a mass ratio of the DHM or the salt of the DHM to the taurine is (2-40):1; and/or a mass ratio of the DHM or the salt of the DHM to the L-theanine is (2-40):1; and/or a mass ratio of the DHM or the salt of the DHM to the cerebroside is (1-40):1.

19. The method according to claim 10, wherein the preparation further comprises a carrier and/or an excipient.

20. The composition for preventing and/or improving the brain dysfunction according to claim 1, wherein the cerebroside is selected from one or more of 1-O-β-D-glucosyl-(2S,3R,4E,8Z)-2-[(2-hydroxyoctadecanoyl)amino]-4,8-octadecadiene-1,3-diol and/or 1-O-β-D-glucosyl-(2S,3R,4E,8Z)-2-[(2-hydroxyhexadecanoyl)amino]-4,8-octadecadiene-1,3-diol.
